(19) United States
(12) Patent Application Publication
(10) Pub. No.: US 2007/0055248 A1
(43) Pub. Date: Mar. 8, 2007

(54) Bone plating system for treatment of hip fractures

(76) Inventors: Michal P. Zlowodzki, Minneapolis, MN (US); Mohit Bhandari, Hamilton (CA)

Correspondence Address: Thomas M. Freiburger, P.O. Box 1026, Tiburon, CA, 94920, US

(21) Appl. No.: 11/192,738

(22) Filed: Jul. 29, 2005

(51) Int. Cl. A61F 2/30

(52) U.S. Cl. 606/69

(57) ABSTRACT

The current invention is a bone plating system comprising a plate and screws. The plate resembles the average shape of the outer surface of the proximal femur (hip region). Cancellous screws through the plate are used to create compression between bone fragments and “locking” screws to create a stable connection between screw and plate. Each screw can accomplish either compression, stable connection with the plate or both. Screw holes in the plate can be slotted, thereby allowing for insertion of screws in relatively different positions to each other with regards to the screw entry points within the plate.

### BACKGROUND OF THE INVENTION

The present invention is an orthopaedic bone plating system and, more particularly a bone plating system for treatment of hip fractures in which the neck of the femur is the site of the fracture.

There is extensive documentation on the successful use of bone plating systems for internal fixation of fractures. Bone plating systems are generally used to attach two bone fragments that are separated by a fracture to both ends of a plate. (FIG. 1) The current invention applies to an entirely different situation in which one of the two main bone fragments can not be attached directly to the plate. (FIG. 2) This situation is typical for many hip fractures e.g. fractures of the femoral neck.

There are principally two different types of screws that are being used in conjunction with bone plating systems: non-locking screws and locking screws. Both can be either cortical screws or cancellous screws. Non-locking screws achieve stability between the bone and the plate by screwing into the bone and exhibiting compression between the bone and the plate or two bone fragments (lag screws). Non-locking screws have no mechanical attachment to the plate. Locking screws are screws that create a fixed connection (“fixed-angle”) between the screw and the plate, thereby offering increased resistance to forces acting on the screw. This concept prevents the screws from changing their position relative to the plate under various smaller loads or at least diminishes the displacement of the screws in relation to the plate under loading. A third type of screws is a combination of the former two: lag screws that can be locked into the plate. Multiple methods are known to achieve fixed-angle stability between the screw and the plate. One possibility is a thread on the screw head that engages into a counter thread in the screw hole of the plate (U.S. Pat. No. 5,709,686, Smith & Nephew PERI-LOC Plating System, and others). Other concepts that are known are based on a friction-fit principle of a screw engaging in a plate where the forces exhibited during the insertion of the screw press an inner ring in the screw hole against the screw hole thereby creating pressure between screw and screw whole resulting in an increased stability (Stryker inc. “Numelock II Polyaxial Locking System” and the DePuy inc. “Polyax Periarticular Plating System”), or a threaded “cap” that engages in a threaded screw hole in the plate after insertion of a screw (Zimmer inc. “NCB Plating System”). In the current invention any type of the above mentioned locking screw mechanisms or any other mechanism can be used.

The cortex of a bone is the outer surface, whereas the “inside” of the bone is comprised of relatively “softer” cancellous bone. Screws can be either cortical or cancellous. Cortical screws gain stability by engaging in the cortex on a bone fragment (“outer” surfaces of the bone). The outer surface of a bone that forms a joint with another bone are generally covered with cartilage. In situations in which it is not desired that the screw “exits/perforates” the outer surface of the bone (Cortex/cartilage; applicable to this invention), so-called cancellous screws are used. Cancellous screws are designed to gain stability in the “softer” “inside” part of the bone. In order to achieve that stability the screw-bone contact surface area is increased by having at least a 1 mm thread or larger (FIG. 3).

Screw holes within a plate can be either “regular” or slotted. Slotted holes are larger lengthy holes that allow for variable positioning of a screw within the plate. Variable positioning of screws allows some flexibility to choose an optimal screw configuration depending on the shape and size of the individual bone and therefore may potentially result in increased stability of the bone-plate-screw construct.

Plate size has an impact on the magnitude of the surgical procedure needed to fix a fracture with a plate. Larger plates require a more invasive procedure to attach the plate to the bone (more muscle stripping etc.). Conversely smaller plates can be used in a less invasive manner (less “cutting” of tissues, less muscle stripping of the bone, etc.) and therefore potentially associated with better treatment results.

Current fixation strategies of femoral neck hip fractures are based the principle of sliding of the femoral head bone fragment along the used implant. The most commonly used implants are multiple parallel cancellous screws without a plate (Synthes “7.3 mm Cannulated Screw Technique Guide” and others) and a so-called sliding hip screws in conjunction with a side plate (U.S. Pat. No. 4,438,762 and others). This principle allows for compression between fracture fragments each time the patient puts weight on the leg. While compression between bone fragments is hypothesized to promote fracture healing, it is associated with potential “shortening” of the femoral neck and therefore a change in the lever arms for many muscles that act on the hip. This change in biomechanics of the hip may have a significant implication on the function of the hip. Also the current devices are associated with a failure risk of up to 30% for some hip fracture types.

Securing the screws to the plate provides a fixed-angle relationship between the plate and screw and reduces the incidence of loosening and femoral neck shortening. As the relationship between the locking screws and the plate is fixed, locking screws provide a high resistance to shear and torsional forces. Also there is some indication that the relative positioning of screws is important for stability of fixation when multiple screws (without a plate) are being used. Thus there exists a need for an improved bone plating system that overcomes the deficiencies of the prior kind.

Several bone plating systems that utilize non-locking and locking screws are known. However, a bone plating system that allows compression of two bone fragments in the region of the proximal femur (hip) with cancellous screws and a fixed stability between screws and plate (locking screws), with or without one or more slotted holes, that allows for variable positioning of screws within a hole of the plate is not available.

### SUMMARY OF THE INVENTION

The current invention is a bone plating system comprised of a plate and multiple screws (FIG. 4 and 5). The plate resembles the shape of the outside surface of the proximal femur (hip region). The bone plating system applies to a situation in which one of the two main bone fragments (1. bone fragment containing the majority of the femoral head and 2. bone fragment in conjunction with the femoral shaft) can not be attached directly to the plate, specifically in fractures of the femoral neck, where the bone fragment containing the femoral head can not be attached to a plate on the outside surface of the femur and can not be perforated by a screw in the area of the femoral head covered with cartilage (FIG. 2).

Typically bone plating systems are used to attach two bone fragments that are separated by a fracture to both ends of a plate (FIG. 1). To achieve this, there have to be at least two screws on either side (total of 4 screws) in line. Therefore the length-to-width ratio of plates is greater than 4, usually even larger. Because in the current invention the one of the two main bone fragments has no direct contact with the plate, the plate can be shorter with a length-to-width ratio greater than 2.5 (FIG. 6). The required surgical procedure can be less invasive when using a smaller plate.

The current invention combines the advantages of fracture compression and fixed-angle screw stability by using lag screws and locking screws. In the presented invention one or more screws have to create compression between the two main bone fragments (1. bone fragment containing the majority of the femoral head and 2. bone fragment in conjunction with the femoral shaft) and the bone fragment adjacent to the plate with the plate. Additionally one or more screws have to result in a fixed mechanical connection (fixed-angle stability) with the plate. Each screw can create either compression between bone fragments or fixed-angle stability or both.

All screws entering the bone fragment that contains the majority of the femoral head (and is separated from the plate by at least one other bone fragment) have to gain stability with the “inside” of the bone and not the opposite cortex/cartilage by “exiting/perforating” the bone. This can be achieved by using so-called cancellous screws that have at least a 1 mm thread or larger (FIG. 3).

Fixed-angle stability with the plate (“locking”) can be achieved with different existing techniques e.g. threaded hole/screw head (FIG. 7), or with any type of technology resulting in a fixed-angle stability between screw and plate (Smith&Nephew “PERI-LOC” Plating System, Stryker inc. “Numelock II Polyaxial Locking System”, Zimmer inc. “NCB Plating System” and others).

One or more screw hole can be a slotted hole that allows for insertion of screws in relatively different positions to each other within the plate and therefore allows for an optimal screw configuration depending on the shape and size of the individual bone.

Screws can be either cannulated or solid. Screws should be ideally partially threaded (smooth screw shaft and threaded screw tip). However, they can also be fully threaded (entire screw threaded with no smooth shaft portion). These and other objects, advantages and features of the invention will be apparent from the following description of a preferred embodiment, considered along with the accompanying drawings.

### DESCRIPTION OF THE DRAWINGS

FIG. 1 depicts a typical situation of a fractured bone in which plates are used.

FIG. 2 depicts a situation of a fractured bone different from a typical situation of a fractured bone in which plates are used. This scenario applies to the current invention.

FIG. 3 depicts a typical cancellous screw.

FIG. 4 depicts a lateral view of the femur with the current invention.

FIG. 5 depicts an anterior view of the femur with the current invention.

FIG. 6 illustrates the dimensions of the plate which is part of the current invention.

FIG. 7 depicts one possible principle of a locking the screw.

### DESCRIPTION OF PREFERRED EMBODIMENTS

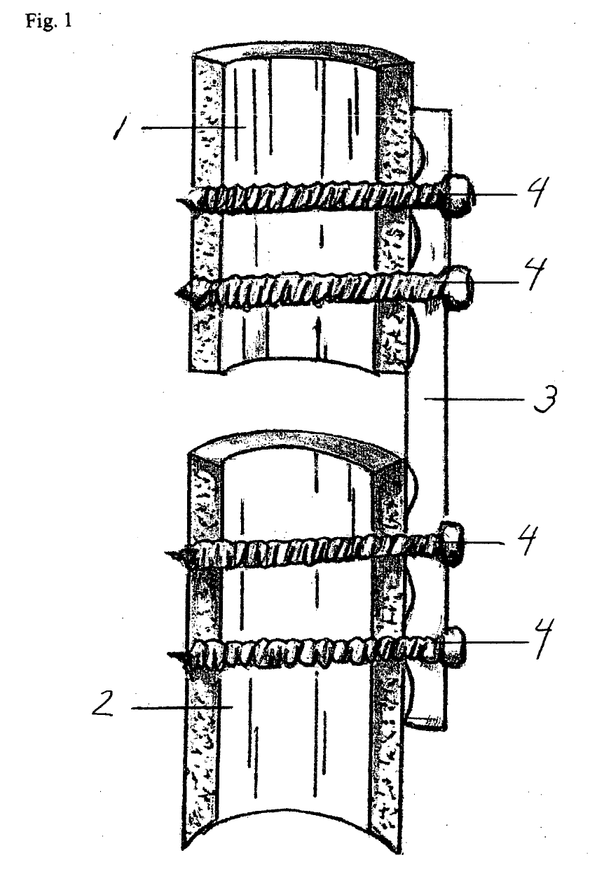

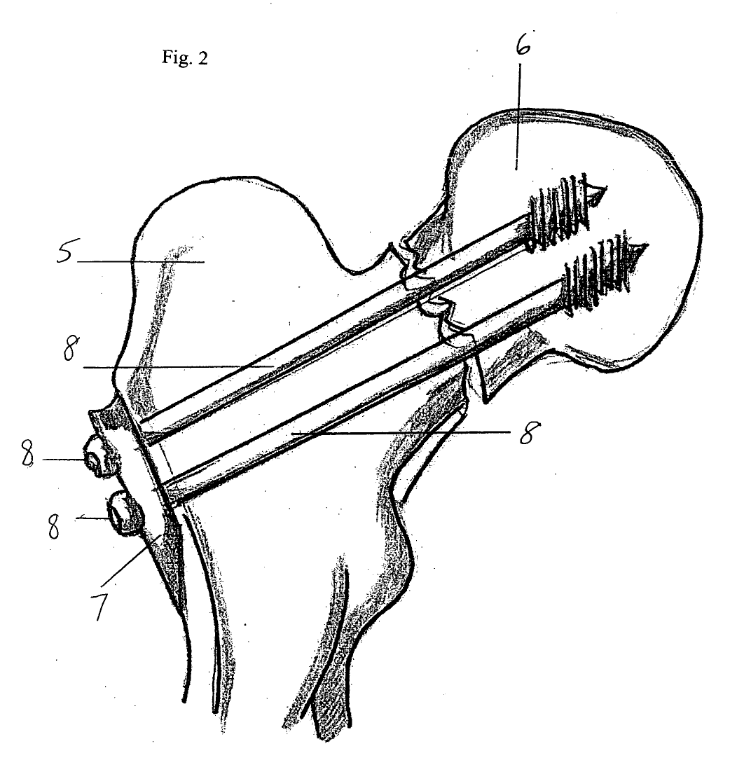

FIG. 2 depicts a situation in which one of the two bone fragments 6 is not adjacent to the plate 7, but separated by another bone fragment 5. Compression between bone fragments is being achieved by multiple screws 8. This situation is applicable to the current invention and fundamentally different from a scenario in which typically plates are used for fixation of a fractured bone depicted in FIG. 1. In the scenario depicted in FIG. 1 both bone fragments 1 and 2 are adjacent to the plate 3 and are being fixed to the plate with separate multiple screws 4. Screws can exert compression between each individual bone fragment 1 and 2 and the plate 3, but not between the bone fragments 1 and 2 themselves.

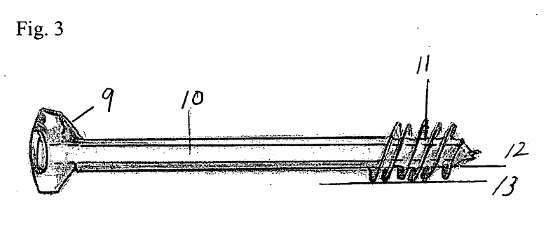

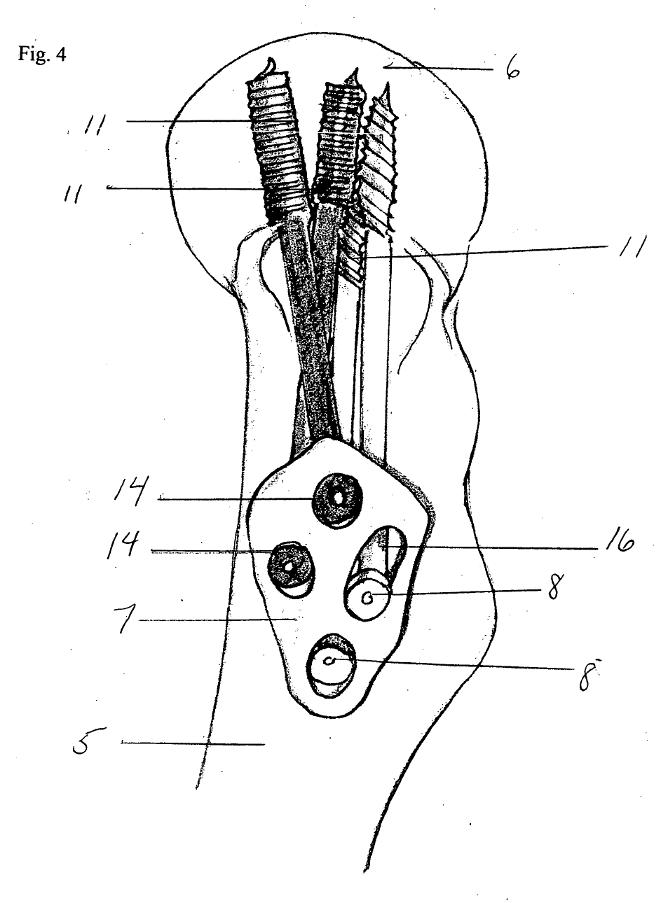

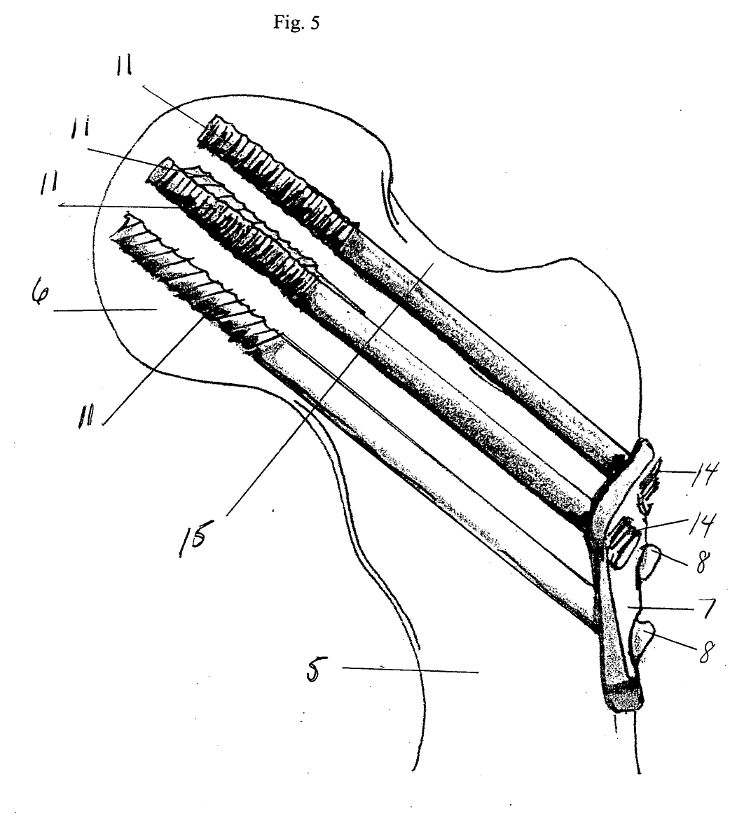

The current invention consists of a plate and multiple cancellous screws. FIG. 3 depicts a typical cancellous screw with a screw head 9 that can be threaded or nonthreaded, a non-threaded screw shaft 10, and a threaded screw tip 11. A cancellous screw can be distinguished from a cortical screw by a larger thread (distance between line 12 and line 13 is at least 1 mm). FIG. 4 depicts a lateral view (side view) of the femur and FIG. 5 an anterior view (front view) of the femur with the current invention. The plate 7 is contoured to resemble the lateral shape of the average upper shaft portion 5 of the femur. The plate has two screw holes that allow for the insertion on non-locking compression screws 8. One of those screw holes has a lengthy shape (slotted hole) 16. There are two holes in the plate 7 that allow for insertion of locking screws 14. All screws have a threaded tip 11 that is inserted in the femoral head 6 which is separated from the shaft portion of the femur 5 by the fracture line 15.

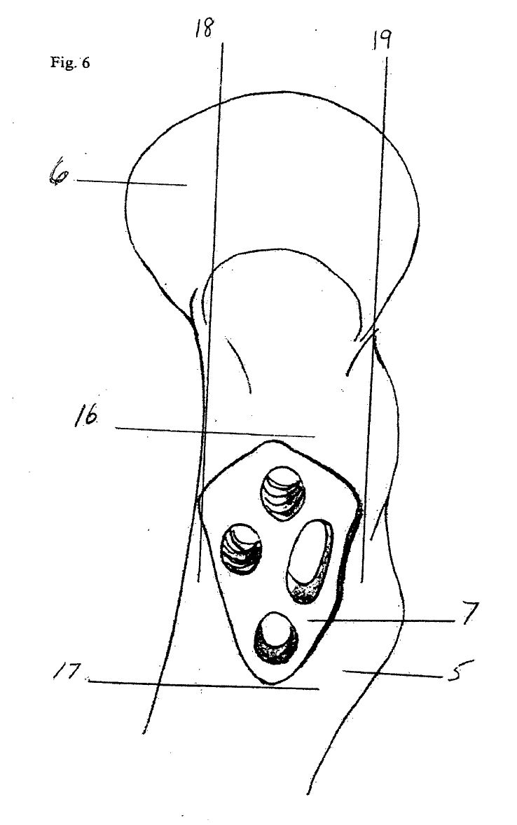

FIG. 6 illustrates the dimensions of the plate. The width of the plate is defined by the distance between the two vertical lines 18 and 19. The length of the plate is defined by the two horizontal lines 16 and 17. The length-to-width ratio is <2.5.

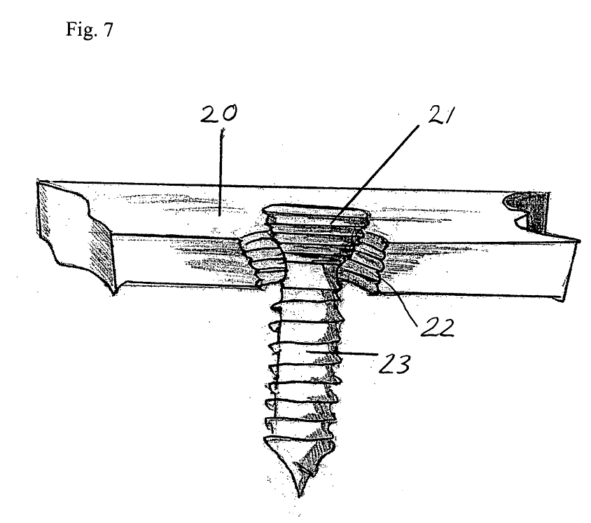

FIG. 7 depicts one possible principle of a locking the screw. The screw has a threaded head 21 that fits into a counter-threaded screw hole 22 of the plate 20.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A bone plating system for fixation of proximal femur (hip) comprising a plate that resembles the surface of the average proximal femur, and has at least one hole for a cancellous lag screw, and at least one hole for a cancellous locking screw.

2. The bone plating system of claim 1, wherein one or more screws are cancellous lag screws that have the option to be locked into the plate.

3. The bone plating system of claim 1, wherein screw locking is achieved with one of the following methods:
a. thread on the screw head and counter-thread in the corresponding screw hole,
b. threaded screw caps and corresponding counter-thread of the inside or outside of the screw hole, and
c. friction-fit between screw and screw hole.

4. The bone plating system of claim 2, wherein screw locking is achieved with one of the following methods:
a. thread on the screw head and counter-thread in the corresponding screw hole,
b. threaded screw caps and corresponding counter-thread of the inside or outside of the screw hole, and
c. friction-fit between screw and screw hole.

5. The bone plating system of claim 1, wherein one or more holes are slotted holes that allow for variable positioning of the screw within the hole.

6. The bone plating system of claim 3, wherein one or more holes are slotted holes that allow for variable positioning of the screw within the hole.

7. The bone plating system of claim 1, wherein the plate is 2-3 cm wide (horizontal), 3-6 cm long (vertical), 1-12 mm thick, and/or the plate dimensions have a length-to-width ratio greater than 2.5.

8. The bone plating system of claim 2, wherein the plate is 2-3 cm wide (horizontal), 3-6 cm long (vertical), 1-12 mm thick, and/or the plate dimensions have a length-to-width ratio greater than 2.5.

9. The bone plating system of claim 3, wherein the plate is 2-3 cm wide (horizontal), 3-6 cm long (vertical), 1-12 mm thick, and/or the plate dimensions have a length-to-width ratio greater than 2.5.

10. The bone plating system of claim 1, wherein there are a total of four holes:
two holes accepting lag screws and two threaded holes accepting locking screws.

11. The bone plating system of claim 7, wherein there are a total of four holes:
two holes accepting lag screws and two threaded holes accepting locking screws.

12. The bone plating system of claim 1, wherein there are a total of four holes:
three holes accepting lag screws that can be locked into the plate and one slotted hole accepting a non-locking lag screw.

13. The bone plating system of claim 3, wherein there are a total of four holes:
three holes accepting lag screws that can be locked into the plate and one slotted hole accepting a non-locking lag screw.

14. The bone plating system of claim 1, wherein the most posterior hole relative to the patient's femur bone is a slotted hole.

15. The bone plating system of claim 12, wherein the most posterior hole relative to the patient's femur bone is a slotted hole.

16. The bone plating system of claim 10, wherein the four holes are located in the corners of the plate.

17. The bone plating system of claim 1, wherein the axis of the holes allows insertion of the screws into the femoral neck.

18. The bone plating system of claim 3, wherein the axis of the holes allows insertion of the screws into the femoral neck.

19. The bone plating system of claim 1, wherein the plate has an extension distally toward the shaft of the femur with holes for one or more locking or non-locking screws.

20. The bone plating system of claim 1, made of steel, titanium, any other metal, metal combination or any other biophysiologically tolerable material.
